(19) United States
(12) Patent Application Publication
(10) Pub. No.: US 2008/0038377 A1
(43) Pub. Date: Feb. 14, 2008

(54) Device for treatment of inflamed tissue

(76) Inventor: Jonathan S. Citow, Glencoe, IL (US)

Correspondence Address: MICHAEL BEST & FRIEDRICH LLP, 100 E WISCONSIN AVENUE, Suite 3300, MILWAUKEE, WI, 53202, US

(21) Appl. No.: 11/501,330

(22) Filed: Aug. 9, 2006

(51) Int. Cl. A61M 31/00; A61K 36/82; A61K 36/38; A61K 36/47; A61K 36/13

(52) U.S. Cl. 424/725; 424/729; 424/771; 424/770; 424/730; 604/500; 424/731

(57) ABSTRACT

A device having a squeezable hollow member connected to a medication chamber in turn connected to a hollow tube fitted with an absorbent tip. The device may be used to conveniently administer a medication to an inflamed tissue. A method of providing a medication to an inflamed tissue is also provided.

### FIELD OF THE INVENTION

The present invention relates generally to medical products and, more particularly, to topical application of suitable compounds for alleviating the symptoms of hemorrhoid irritation and other anorectal diseases.

### BACKGROUND OF THE INVENTION

Many chemical compounds are disclosed in the literature for treatment of inflamed hemorrhoidal tissue and other anorectal diseases. Prior art medicines for hemorrhoidal diseases include analgesic/antiphlogistic agents, hemostatic agents, astringents, disinfectants, etc. Some compounds have the benefit of reducing itching, soreness and other side effects associated with inflamed anorectal tissue. Examples of such compounds include zinc peroxide and urea hydrogen peroxide or benzyl peroxide in combination with hydrocortisone. Lidocaine has been used for application for rectal irritation (U.S. Pat. No. 6,740,333). Typically, these preparations are provided in the form of suppositories having a semi-solid consistency at room temperature such that they can be wrapped in foil and administered manually.

A disadvantage of common preparations sold in suppository form for the treatment of hemorrhoids or other anorectal conditions is that they melt when their temperature is raised, such as by grasping them with the fingers, thereby leaving residue on the fingers which the user typically must remove as by hand washing with soap and water, for example. Alternatively, the user might employ a surgical glove to administer the suppository then discard the glove when the process in complete. In either event, the process of administering a suppository of the type commonly used to treat inflamed hemorrhoidal tissue can be an inconvenient and time consuming process.

Topical applicators are known for dispensing medicinal and therapeutic agents (U.S. Pat. Nos. 5,738,643, 5,762,494, 5,378,226 and 4,887,994) but in these, the delivery tip requires first to be impregnated with the medicinal and therapeutic agent.

### SUMMARY OF THE INVENTION

A device for treatment of an inflamed hemorrhoidal tissue or of an anorectal area is provided comprising: a squeezable hollow member connected to a medication chamber adapted to store and discharge a medicinal or therapeutic agent, the medication chamber in turn connected to a hollow tube adapted for passage of the agent, the hollow tube being connected to an absorbent tip, wherein when said hollow member is squeezed the agent is caused to infuse said tip. In one embodiment, the hollow tube further comprises a membrane adapted to rupture when said hollow member is squeezed, wherein when the membrane is rendered permeable by rupture the agent is caused to infuse said tip. In another embodiment, the device includes a topical solution of a lidocaine compound. In yet other embodiments, one or more of the hollow member, the medication chamber and the hollow tube is formed from a break-resistant plastic. In yet further embodiments, the tip is formed of a substance selected from the group consisting of cotton and sponge.

The medicinal or therapeutic agent may be one, or a mixture or combination of the following: tetracycline, doxycycline, oxytetracylcline, chloramphenicol, erythromycin, acyclovir, idoxuridine, tromantadine, miconazole, ketoconazole, fluconazole, itraconazole, econazole, griseofulvin, amphotericin B, nystatine, metronidazole, metronidazole benzoate, tinidazole, indomethacin, ibuprofen, piroxicam, diclofenac, disodium cromoglycate, nitroglycerin, isosorbide dinitrate, verapamile, nifedipine, diltiazem, digoxine, morphine, cyclosporins, buprenorphine, benzocaine, lidocaine, procaine, diazepam, nitrazepam, flurazepam, estazolam, flunitrazepam, triazolam, alprazolam, midazolam, temazepam lormetazepam, brotizolam, clobazam, clonazepam, lorazepam, oxazepam, busiprone, sumatriptan, ergotamine derivatives, cinnarizine, antihistamines, ondansetron, tropisetron, granisetrone, metoclopramide, disulfiram, vitamin K, cortisone, hydrocortisone, betamethasone, dexamethasone, fluocortolone, prednisolone, triamcinolone, indomethacin, sulindac, sulindac salts, corresponding sulindac sulfide, beta-lactam antibiotics, chloramphenicol, neomycin, gramicidin, bacitracin, sulfonamides, aminoglycoside antibiotics, tobramycin, nitrofurazone, nalidixic acid and analogs, the antimicrobial combination of fludalanine/pentizidone, potassium channel mediating, regulating or blocking agents, calcium channel blocking or regulatory agents, sodium channel blocking agents, steroids, non-steroidal anti-inflammatory agents, aloe vera, chamomile, α-bisabolol, cola nitada extract, green tea extract, tea tree oil, licorice extract, allantoin, urea, caffeine and other xanthenes, and glycyrrhizic acid and its derivatives Acidum muriaticum (hydrochloric acid), Aesculus hippocastanum (horse chesnut), Alcloxa, aluminum hydroxide gel, Arctium lappa, benzyl, Bryonia alba (white bryony), calamine, Calcarea fluorica (calcium fluoride), Calcarea phosphorica (phosphate of calcium), Calendula officinalis, camphor, cocoa butter, cod liver oil, Collinsonia canadensis (stone-root), dibucaine hydrochloride, dibucaine, dyclonine hydrochloride, Echinacea, ephedrine sulfate, epinephrine hydrochloride, epinephrine, Ferrum phosphoricum (phosphate of iron), glycerin, graphites (black lead), Hamamelis virginiana, hard fat, Hydrastis canadensis, Hypericum, juniper tar, Kali carbonicum (Potassium carbonate), Kali phosphoricum (phosphate of potassium), kaolin, lanolin, Lycopodium clavatum (clubmoss), lycopodium, magnesia phosphorica (phosphate of magnesium), menthol, millefolium (yellow dock), mineral oil, Natrum phosphoricum (phosphate of soda), Nitricum acidum (nitric acid), Nux vomica (Quaker Button), Paeonia officinalis (peony), petrolatum, phenylephrine hydrochloride, plantago, pramoxine hydrochloride, Quercus, Ratanhia, resorcinol, resorcinum, solidago, sulphur (sublimed sulphur), tetracaine hydrochloride, tetracaine, topical starch, Urtica urens, Viburnum opulus, white petrolatum, witch hazel, zinc oxide, mixtures thereof and a combination thereof, optionally in a pharmaceutically acceptable carrier selected from the group consisting of: acetone sodium bisulfite, alcohol, almond oil, aloe barbadensis extract, aloe barbadensis gel, anhydrous lanolin, ascorbyl palmitate, avocado Oil, beeswax, benzethonium chloride, benzoic acid, benzophenone-4; benzyl alcohol, benzyl benzoate, calcium phosphate dibasic, canola oil, capryl/capramidopropyl betaine, carboxymethylcellulose sodium, castor oil, cetyl alcohol, citric acid, cocoa butter, corn oil, cottonseed oil, diazolidinyl urea, edetate disodium; glycerin, glyceryl monooleate, glyceryl monostearate, glyceryl oleate, glyceryl stearate, grain alcohol, hydrogenated vegetable oil, hydroxyethylcellulose; kaolin, lactose, lanolin, lanolin alcohol, light mineral oil, magnesium stearate, methylparaben, microcrystalline wax, mineral oil, olive, olive oil, palm fruit oil, palm, palm oil, paraffin, PEG 12 dilaurate, peruvian balsam, petrolatum, petroleum, polyethylene gycol 300, polyethylene wax, polysorbate 80, propyl gallate; propylene glycol, propylparaben, purified water; shark liver oil; simethicone; sodium benzoate, sodium citrate, sodium lauryl sulfate, sorbitan sesquioleate, soybean oil, sucrose, starch, stearyl alcohol, sweet almond oil, thyme oil, tocopherol; tocopheryl acetate, trisodium EDTA, vitamin E, wax, white petrolatum, white wax, xanthan gum, mixtures thereof and a combination thereof.

Furthermore, a kit is provided for treatment of inflamed hemorrhoidal tissue or anorectal disease, comprising a device comprising a squeezable hollow member connected to a medication chamber adapted to store and discharge a therapeutic agent, the medication chamber in turn connected to a hollow tube adapted for passage of the agent, the hollow tube being connected to an absorbent tip, wherein when said hollow member is squeezed the agent is caused to infuse said tip, the device being enclosed in a frangible wrapper.

In addition, a method of treatment of an inflamed hemorrhoidal tissue or of anorectal disease is provided, the method comprising the steps of: providing a device comprising a squeezable hollow member connected to a medication chamber adapted to store and discharge a therapeutic agent, the medication chamber in turn connected to a hollow tube adapted for passage of the agent, the hollow tube being connected to an absorbent tip, wherein when said hollow member is squeezed the agent is caused to infuse said tip; squeezing said squeezable hollow member; and then applying said tip to an inflamed hemorrhoidal tissue or anorectal region.

The applicator can thus be used to be applied to a subject's inflamed hemorrhoidal tissue or of an anorectal area. When the applicator is packaged, it may be maintained in a generally sterile environment until the time of use, the risk of contamination of the anesthetic-cleansing solution used is greatly reduced.

### BRIEF DESCRIPTION OF DRAWINGS

The foregoing and other novel features and advantages of the invention will be better understood upon a reading of the following detailed description taken in conjunction with the accompanying drawing wherein:

FIG. 1 is a perspective view of an embodiment of the device according to the invention.

FIG. 2 is an alternative embodiment of the inventive device showing a breakable membrane situated in the hollow tube between the medication chamber and the absorbent tip.

FIG. 3 is a view of the device packaged in a frangible pouch.

### DETAILED DESCRIPTION

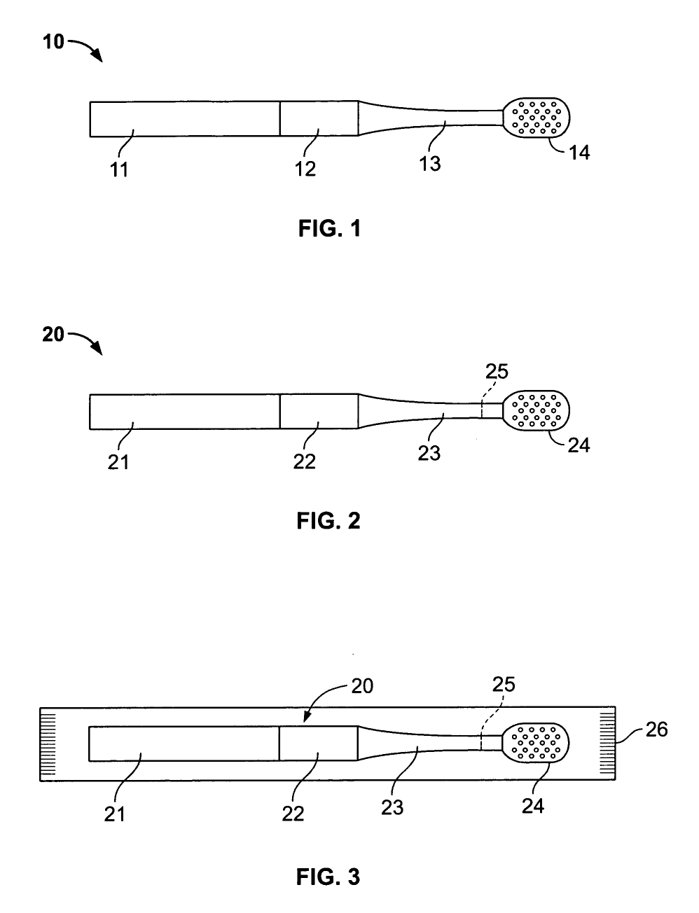

Referring now to the drawings, FIG. 1 shows a device designated generally by the reference numeral 10, for administering a topical therapeutic or medicinal agent or preparation to an inflamed hemorrhoidal tissue, or an anorectal area, for example. The device includes as its principal components squeezable hollow member 11, connected to medication chamber 12, the medication chamber in turn connected to hollow tube 13, the hollow tube being in turn connected to absorbent tip 14.

FIG. 2 shows an alternative embodiment designated generally by the reference numeral 20, in which squeezable hollow member 21 is connected to medication chamber 22, the medication chamber in turn connected to hollow tube 23, the hollow tube being in turn connected to absorbent tip 24, and breakable membrane 25 is situated in hollow tube 23 between medication chamber 22 and absorbent tip 24. In device 20, the required squeeze pressure of device 20 necessary to deliver the agent will depend on the effective burst pressure and tensile strength of the membrane 25. Thus, the performance of the device 20 may be varied by appropriately selecting and configuring the material of the membrane to provide the required effective burst pressure and tensile strength of the membrane.

FIG. 3 shows a device according to the invention illustrated in FIG. 2 encased within an exemplary suitable packaging 26.

It can be appreciated that a device according to the invention offers considerable convenience in the administering of topical preparations, particularly to inflamed hemorrhoidal tissue, and offers considerable advantages over the use of suppositories, for example. Device 10 or 20 can be grasped by squeezable hollow member 21 and squeezed to apply the preparation without exposing the user's fingers to the preparation. The device 10 or 20 when packaged can very easily be removed from its wrapper 26. Once the application process is completed, the device may simply be discarded without the need for any clean-up.

In one embodiment, the topical medicinal agent or preparation comprises a lidocaine compound, such as a topical solution of about 4% Lidocaine. Hydrochloride; a cream, gel, or ointment may also be used. This preparation may also be mixed with anti-inflammatories, such as steroids. The squeezable hollow member 21 may be on the order of 5 cm long so as to be convenient to carry the device 10 or 20 in a purse or wallet.

Thus, the device in accordance with the invention can be quickly and easily used to be applied to a subject's inflamed hemorrhoidal tissue, or anorectal area. Since the device 10 or 20 may be maintained in a generally sterile environment when packaged up till the time of use, the risk of contamination of the anesthetic-cleansing solution used is substantially reduced.

The terms “anorectal region” or “anorectal area” should be construed herein to include the anus and the rectum, as well as all the regions of the anus, internal as well as external, such as and without limitation: the anal crypt, the anal gland, the external sphincter and the venous plexus below the dentate line, and also all the regions of the rectum such as and without limitation: the internal sphincter and venous plexus above the dentate line.

It will be understood that many different medicinal or therapeutic agents may be used in the inventive device not limited to topical anesthetics, agonists, anti-inflammatories, antibiotics, homeopathics or cleaning agents, and the like, and mixtures thereof. The medicinal or therapeutic agent may be selected from the agent is selected from the group consisting of: tetracycline, doxycycline, oxytetracylcline, chloramphenicol, erythromycin, acyclovir, idoxuridine, tromantadine, miconazole, ketoconazole, fluconazole, itraconazole, econazole, griseofulvin, amphotericin B, nystatine, metronidazole, metronidazole benzoate, tinidazole, indomethacin, ibuprofen, piroxicam, diclofenac, disodium cromoglycate, nitroglycerin, isosorbide dinitrate, verapamile, nifedipine, diltiazem, digoxine, morphine, cyclosporins, buprenorphine, benzocaine, lidocaine, procaine, diazepam, nitrazepam, flurazepam, estazolam, flunitrazepam, triazolam, alprazolam, midazolam, temazepam lormetazepam, brotizolam, clobazam, clonazepam, lorazepam, oxazepam, busiprone, sumatriptan, ergotamine derivatives, cinnarizine, anti-histamines, ondansetron, tropisetron, granisetrone, metoclopramide, disulfiram, vitamin K, cortisone, hydrocortisone, betamethasone, dexamethasone, fluocortolone, prednisolone, triamcinolone, indomethacin, sulindac, sulindac salts, corresponding sulindac sulfide, beta-lactam antibiotics, chloramphenicol, neomycin, gramicidin, bacitracin, sulfonamides, aminoglycoside antibiotics, tobramycin, nitrofurazone, nalidixic acid and analogs, the antimicrobial combination of fludalanine/pentizidone, potassium channel mediating, regulating or blocking agents, calcium channel blocking or regulatory agents, sodium channel blocking agents, steroids, non-steroidal anti-inflammatory agents, aloe vera, chamomile, α-bisabolol, cola nitada extract, green tea extract, tea tree oil, licorice extract, allantoin, urea, caffeine and other xanthenes, and glycyrrhizic acid and its derivatives Acidum muriaticum (hydrochloric acid), Aesculus hippocastanum (horse chesnut), Alcloxa, aluminum hydroxide gel, Arctium lappa, benzyl, Bryonia alba (white bryony), calamine, Calcarea fluorica (calcium fluoride), Calcarea phosphorica (phosphate of calcium), Calendula officinalis, camphor, cocoa butter, cod liver oil, Collinsonia canadensis (stone-root), dibucaine hydrochloride, dibucaine, dyclonine hydrochloride, Echinacea, ephedrine sulfate, epinephrine hydrochloride, epinephrine, Ferrum phosphoricum (phosphate of iron), glycerin, graphites (black lead), Hamamelis virginiana, hard fat, Hydrastis canadensis, Hypericum, juniper tar, Kali carbonicum (Potassium carbonate), Kali phosphoricum (phosphate of potassium), kaolin, lanolin, Lycopodium clavatum (clubmoss), lycopodium, magnesia phosphorica (phosphate of magnesium), menthol, millefolium (yellow dock), mineral oil, Natrum phosphoricum (phosphate of soda), Nitricum acidum (nitric acid), Nux vomica (Quaker Button), Paeonia officinalis (peony), petrolatum, phenylephrine hydrochloride, plantago, pramoxine hydrochloride, Quercus, Ratanhia, resorcinol, resorcinum, solidago, sulphur (sublimed sulphur), tetracaine hydrochloride, tetracaine, topical starch, Urtica urens, Viburnum opulus, white petrolatum, witch hazel, zinc oxide, mixtures thereof and a combination thereof, optionally in a pharmaceutically acceptable carrier selected from the group consisting of: acetone sodium bisulfite, alcohol, almond oil, aloe barbadensis extract, aloe barbadensis gel, anhydrous lanolin, ascorbyl palmitate, avocado Oil, beeswax, benzethonium chloride, benzoic acid, benzophenone-4; benzyl alcohol, benzyl benzoate, calcium phosphate dibasic, canola oil, capryl/capramidopropyl betaine, carboxymethylcellulose sodium, castor oil, cetyl alcohol, citric acid, cocoa butter, corn oil, cottonseed oil, diazolidinyl urea, edetate disodium; glycerin, glyceryl monooleate, glyceryl monostearate, glyceryl oleate, glyceryl stearate, grain alcohol, hydrogenated vegetable oil, hydroxyethylcellulose; kaolin, lactose, lanolin, lanolin alcohol, light mineral oil, magnesium stearate, methylparaben, microcrystalline wax, mineral oil, olive, olive oil, palm fruit oil, palm, palm oil, paraffin, PEG 12 dilaurate, peruvian balsam, petrolatum, petroleum, polyethylene gycol 300, polyethylene wax, polysorbate 80, propyl gallate; propylene glycol, propylparaben, purified water; shark liver oil; simethicone; sodium benzoate, sodium citrate, sodium lauryl sulfate, sorbitan sesquioleate, soybean oil, sucrose, starch, stearyl alcohol, sweet almond oil, thyme oil, tocopherol; tocopheryl acetate, trisodium EDTA, vitamin E, wax, white petrolatum, white wax, xanthan gum, mixtures thereof and a combination thereof.

It may be desirable to mix such agents with a biocompatible material such as for example a polymeric substance or another agent for extending the release time of the active ingredient(s). One example of a medicational or therapeutic agent suited to the inventive device is a composition containing an agonist with a penetration enhancer, such as is described in U.S. Patent Application 20050090557, the contents of which are incorporated herein. Other examples of such suitable compositions is described in U.S. Patent Application 20030124202, the contents of which are incorporated herein.

The term “pharmaceutically acceptable carrier” or “pharmaceutically acceptable vehicle” should be construed herein to include materials that are suitable for anorectal, external, intra-anal or intra-rectal or topical or local administration to the anorectal region that are not biologically or otherwise undesirable, i.e., that may be administered to an individual along with an active agent without causing any undesirable biological effects or interacting in a deleterious manner with any of the other components of the anorectal formulation in which it is contained.

The present invention has been described in the foregoing specification with respect to specific embodiments. These embodiments serve as examples to illustrate the invention rather than to limit its scope. Modifications may be made thereto without departing from the broader teachings of the invention.

What is claimed is:

1. A device for treatment of an inflamed hemorrhoidal tissue or anorectal area, comprising: a squeezable hollow member connected to a medication chamber adapted to store and discharge a therapeutic agent, the medication chamber in turn connected to a hollow tube adapted for passage of the agent, the hollow tube being connected to an absorbent tip, wherein when said hollow member is squeezed the agent is caused to infuse said tip.

2. The device of claim 1, wherein said hollow tube further comprises a breakable membrane adapted to rupture when said hollow member is squeezed.

3. The devices of claims 1 or 2, wherein the agent is selected from the group consisting of: tetracycline, doxycycline, oxytetracylcline, chloramphenicol, erythromycin, acyclovir, idoxuridine, tromantadine, miconazole, ketoconazole, fluconazole, itraconazole, econazole, griseofulvin, amphotericin B, nystatine, metronidazole, metronidazole benzoate, tinidazole, indomethacin, ibuprofen, piroxicam, diclofenac, disodium cromoglycate, nitroglycerin, isosorbide dinitrate, verapamile, nifedipine, diltiazem, digoxine, morphine, cyclosporins, buprenorphine, benzocaine, lidocaine, procaine, diazepam, nitrazepam, flurazepam, estazolam, flunitrazepam, triazolam, alprazolam, midazolam, temazepam lormetazepam, brotizolam, clobazam, clonazepam, lorazepam, oxazepam, busiprone, sumatriptan, ergotamine derivatives, cinnarizine, antihistamines, ondansetron, tropisetron, granisetrone, metoclopramide, disulfiram, vitamin K, cortisone, hydrocortisone, betamethasone, dexamethasone, fluocortolone, prednisolone, triamcinolone, indomethacin, sulindac, sulindac salts, corresponding sulindac sulfide, beta-lactam antibiotics, chloramphenicol, neomycin, gramicidin, bacitracin, sulfonamides, aminoglycoside antibiotics, tobramycin, nitrofurazone, nalidixic acid and analogs, the antimicrobial combination of fludalanine/pentizidone, potassium channel mediating, regulating or blocking agents, calcium channel blocking or regulatory agents, sodium channel blocking agents, steroids, non-steroidal anti-inflammatory agents, aloe vera, chamomile, α-bisabolol, cola nitada extract, green tea extract, tea tree oil, licorice extract, allantoin, urea, caffeine and other xanthenes, and glycyrrhizic acid and its derivatives Acidum muriaticum (hydrochloric acid), Aesculus hippocastanum (horse chesnut), Alcloxa, aluminum hydroxide gel, Arctium lappa, benzyl, Bryonia alba (white bryony), calamine, Calcarea fluorica (calcium fluoride), Calcarea phosphorica (phosphate of calcium), Calendula officinalis, camphor, cocoa butter, cod liver oil, Collinsonia canadensis (stone-root), dibucaine hydrochloride, dibucaine, dyclonine hydrochloride, Echinacea, ephedrine sulfate, epinephrine hydrochloride, epinephrine, Ferrum phosphoricum (phosphate of iron), glycerin, graphites (black lead), Hamamelis virginiana, hard fat, Hydrastis canadensis, Hypericum, juniper tar, Kali carbonicum (Potassium carbonate), Kali phosphoricum (phosphate of potassium), kaolin, lanolin, Lycopodium clavatum (clubmoss), lycopodium, magnesia phosphorica (phosphate of magnesium), menthol, millefolium (yellow dock), mineral oil, Natrum phosphoricum (phosphate of soda), Nitricum acidum (nitric acid), Nux vomica (Quaker Button), Paeonia officinalis (peony), petrolatum, phenylephrine hydrochloride, plantago, pramoxine hydrochloride, Quercus, Ratanhia, resorcinol, resorcinum, solidago, sulphur (sublimed sulphur), tetracaine hydrochloride, tetracaine, topical starch, Urtica urens, Viburnum opulus, white petrolatum, witch hazel, zinc oxide, mixtures thereof and a combination thereof, optionally in a pharmaceutically acceptable carrier selected from the group consisting of: acetone sodium bisulfite, alcohol, almond oil, aloe barbadensis extract, aloe barbadensis gel, anhydrous lanolin, ascorbyl palmitate, avocado Oil, beeswax, benzethonium chloride, benzoic acid, benzophenone-4; benzyl alcohol, benzyl benzoate, calcium phosphate dibasic, canola oil, capryl/capramidopropyl betaine, carboxymethylcellulose sodium, castor oil, cetyl alcohol, citric acid, cocoa butter, corn oil, cottonseed oil, diazolidinyl urea, edetate disodium; glycerin, glyceryl monooleate, glyceryl monostearate, glyceryl oleate, glyceryl stearate, grain alcohol, hydrogenated vegetable oil, hydroxyethylcellulose; kaolin, lactose, lanolin, lanolin alcohol, light mineral oil, magnesium stearate, methylparaben, microcrystalline wax, mineral oil, olive, olive oil, palm fruit oil, palm, palm oil, paraffin, PEG 12 dilaurate, peruvian balsam, petrolatum, petroleum, polyethylene gycol 300, polyethylene wax, polysorbate 80, propyl gallate; propylene glycol, propylparaben, purified water; shark liver oil; simethicone; sodium benzoate, sodium citrate, sodium lauryl sulfate, sorbitan sesquioleate, soybean oil, sucrose, starch, stearyl alcohol, sweet almond oil, thyme oil, tocopherol; tocopheryl acetate, trisodium EDTA, vitamin E, wax, white petrolatum, white wax, xanthan gum, mixtures thereof and a combination thereof.

4. The device of claim 3, wherein the agent includes a topical solution of about 4% of a lidocaine compound.

5. The device of claim 3, wherein said hollow member is formed from a break-resistant plastic.

6. The device of claim 3, wherein said medication chamber is formed from a break-resistant plastic.

7. The device of claim 3, wherein said hollow tube is formed from a break-resistant plastic.

8. The device of claim 3, wherein said tip is formed of a substance selected from the group consisting of cotton and sponge.

9. The device of claim 3, wherein said squeezable hollow member is approximately 5 cm in length.

10. A kit for treatment of an inflamed hemorrhoidal tissue or anorectal area, comprising the device of claim 3 being packaged in a frangible wrapper.

11. A method of treatment of an inflamed hemorrhoidal tissue or anorectal area, comprising the steps of:
(a) providing the device of claim 3;
(b) squeezing said squeezable hollow member; and then
(c) applying said tip to an inflamed hemorrhoidal tissue or anorectal region.
